(19) United States
(12) Patent Application Publication
(10) Pub. No.: US 2004/0242986 A1
(43) Pub. Date: Dec. 2, 2004

(54) Hand held tonometer with optical arrangement for indicating critical distance fron an eye

(76) Inventors: James Robert Arnold Matthews, Berkshire (GB); John Horace Fisher, Hertfordshire (GB); Paul Antony Merritt, Hertfordshire (GB)

Correspondence Address: BARNES & THORNBURG, P.O. BOX 2786, CHICAGO, IL, 60690-2786, US

(21) Appl. No.: 10/486,418

(22) PCT Filed: Aug. 13, 2002
(86) PCT No.: PCT/GB02/03733

(30) Foreign Application Priority Data
Aug. 14, 2001 (GB) 0119741.7
Aug. 14, 2001 (GB) 0119743.3
Aug. 14, 2001 (GB) 0119744.1

(51) Int. Cl. A61B 3/16

(52) U.S. Cl. 600/401

(57) ABSTRACT

A puff tonometer which includes an object at a point in the optical path of light from a source of light in the tonometer, typically near to the source, such that an in-focus image of the object will be formed in the user's field of view when the tonometer is at the critical distance from an eye under test at which the automatic air pulse generating means will be triggered by light reflected from the eye forming an in focus image of a mask on a plurality of photoelectric sensors. A second object may be located in the same region of the tonometer as the first object, but in a plane which is spaced from the plane containing the first object, whereby its image will come into focus in the field of view just before the image of the first object comes into focus, as the unit is moved slowly towards the patient's eye. A third object visually distinguishable from both the first and the second objects may be located in a third plane spaced from the plane containing the first object, whereby its image will come into focus if the unit is moved closer to the patient's eye than said critical distance. Two supplementary light sources may be provided on opposite sides of the puff tube of the tonometer to project two narrow beams of light towards an eye under test, to appear as two spots of light in the field of view ahead of when the in focus image of the object(s) will be seen. Typically the latter is formed using red light and the two supplementary light sources produce green light. A longer focal length eyepiece may be employed to allow an image of the eye to be seen from a distance to assist in initially lining up the tonometer with the patient's face.

### FIELD OF INVENTION

This invention relates to a non-contact air impulse tonometer of the type in which a controlled pulse of air is directed towards the cornea of an eye under test and the resulting momentary deformation of the cornea monitored, to determine the internal pressure of the eye relative to the ambient, and indicate the monitored pressure to the user.

### BACKGROUND TO THE INVENTION

An air impulse tonometer which can be held in the hand in use, is described in UK 2175412 and EP 0289545. Such a tonometer will be referred to as a tonometer of the type described.

Initial alignment of such a tonometer with an eye under test, can be difficult since the optical system developed for that tonometer includes an eyepiece which does not allow an image of the eye under test to be seen by the user when looking through the eyepiece. Instead a filament lamp and red filter (which comprise a source of illumination), a condenser lens and objective lens assembly, project red light through a mask (containing two windows but otherwise obscured) towards the eye. At one particular distance between the eye under test and the objective lens assembly, the convex anterior surface of the cornea of the eye and the objective lens form an in-focus image of the two windows which can be seen by a user looking through the eyepiece, the windows appearing as two separate red segments. Since the focus of the red light is determined by the distance between the optical system in the hand held unit and the anterior corneal surface of the eye under test (from which it is reflected), movement of the unit towards and away from the eye will alter the focus of the two segments of red light as seen by the user, thereby assisting the user in positioning the unit relative to the eye.

The sensing mechanism is set up to instigate an air pulse when the reflected light is centred on the optical axis and an image of the mask is in focus on a plurality of photoelectric sensors and each receive preselected amounts of the reflected light. This also corresponds to the position of the unit relative to the eye at which the two illuminated segments are in focus in the field of view.

In practice the user will tend to look along the side of the unit as he/she moves the unit into position until he/she is satisfied that, from experience, the unit is nearly close enough to the eye to allow the measurement to be taken. At this point the user can now look through the eyepiece of the unit to view the image in the field of view, as described above, to position the unit into the firing position.

If the user moves the unit closer to the eye before the air pulse is initiated, the two illuminated segments (typically of red light) will begin to go out of focus again (having previously become in-focus at the correct distance), and further movement of the unit towards the eye will result in the filament of the lamp coming into focus in the field of view. Should this happen the user knows to move the unit backwards until the correct point of focus is achieved once again, whereupon it may be necessary to move the unit from side to side or up and down to centre it on the eye, before the unit will fire.

### OBJECT OF THE INVENTION

It is an object of the present invention to provide a mechanism by which the user is assisted in positioning the unit relative to the eye of a patient, so as to cause the unit to trigger and fire a puff of air towards the eye.

### SUMMARY OF THE INVENTION

According to the present invention in a tonometer of the type described an object is placed in the optical path of light from the source of illumination in the tonometer such that an in-focus image of the object will be formed in the user's field of view when the unit is at the critical distance from the eye under test at which the automatic air pulse generating means will be triggered.

Typically the object is placed near to the said source and may be an opaque “hairline” pattern in a transparent support, or the pattern may be formed from a photographic image on a sheet of glass or plastics material or from an etched metal film on a sheet of glass or plastics, or it may be formed by etching a metal foil or from wire(s).

Typically the pattern comprises at least one line which extends in a plane generally perpendicular to the axis along which light is projected from the said source.

The pattern may for example comprise a planar array such as a single line, two lines which cross at an angle, a circular outline with two or more radial lines, or a spiral.

A second object which may be any of the above may be located in the same region of the tonometer as the first object, albeit in a plane which is spaced from the plane containing the first object, on that side thereof which will come into focus in the field of view just before the first object comes into focus, as the unit is moved slowly towards the patient's eye.

Preferably the second object comprises a pattern which is visually distinguishable (as by orientation or content) from the first.

Thus if the objects are single lines and the line which comes into focus at the firing position appears vertical, the wire which is to come into focus earlier is preferably arranged so that it will appear horizontal, or vice versa.

Alternatively if the first object comprises a pair of lines which cross at an angle (say 45° to define a letter X) the second object may comprise a pair of lines which cross at right angles and define a cross, one limb of which is vertical and the other is horizontal.

A third object may also be provided, again preferably distinguishable from both the first and the second objects, at a position relative to the source of illumination such that its image will come into focus if the unit is moved closer to the eye than the critical firing position.

In a tonometer of the type described which includes one or more objects as aforesaid the eyepiece can be redesigned to give a lower magnification which will give an in-focus image of the patient's eye at a distance via the objective lens. However, at closer distances, but greater than that at which the instrument will fire, the image of the eye goes out of focus and the field of view becomes less than the normal diameter of the patient's pupil so the alignment cues for the user of the instrument disappear and only darkness can be seen in the field of view.

With this re-designed eyepiece, it is no longer possible for the user to see an in-focus image of the window mask as the instrument comes into the correct alignment position for the air-puff to be discharged. It is therefore necessary to provide additional alignment aids, one of which is the use of an object or objects, positioned as described above, so that the image, formed from the anterior surface of the cornea of the eye being tested, the objective lens and an eyepiece, comes into view at close quarters.

A tonometer incorporating the re-designed lower magnification eyepiece allows the user to look through the eyepiece and identify the patient's eye to be tested, whilst at some distance from the patient's face.

Thereafter the user can move the unit towards the eye, keeping the image of the eye in the centre of the field of view. As the distance between the unit and the patient's eye decreases, light (typically red light), reflected from the anterior surface of the cornea will be seen to fill the windows of the mask, and this then forms an additional positioning aid.

As the modified unit is then moved further towards the firing position the image of the, or each of the objects, as aforesaid, will be seen, and these can be aligned and focused by moving the unit, so that it is finally in the correct position to fire.

Hitherto when using a tonometer of the type described, the user has had to view the patient's eye which is to be tested, by looking along the side of the unit prior to adjusting their viewing position to look through the unit. Instead the user can now continue to look through the eyepiece of the unit all the time.

In a unit embodying the invention the eyepiece and objective lens form a simple telescope with an inverted image. A Pechan-Schmidt prism may therefore be located between the eyepiece lens and a window through which the user looks, to invert the image and present to the user an image of the patient's eye which is correctly oriented and handed in a vertical and horizontal sense.

The focal length of the eyepiece is preferably in the range 62-100 mm, typically 80 mm.

In a tonometer embodying the invention but modified so as to enable the user to view the patient's eye as aforesaid, the image of the patient's pupil will disappear as the unit is moved closer to the patient, and shortly before the unit is close enough for light from the source of illumination in the tonometer to be reflected from the anterior corneal surface to illuminate the mask, and be seen in the eyepiece as two illuminated mask windows. It is therefore preferable to provide an additional positioning or alignment aid to bridge the gap between being able to see the pupil and being able to see the illuminated mask windows.

As a preferred approach a tonometer incorporating one or more objects as aforesaid which will come into focus at or near the critical distance at which the tonometer is to fire, may also include two small light sources at diametrically opposite points, typically equidistant from the optical axis of the objective lens assembly, such that in use and positioned close to a patient's eye, light from the two sources, after reflection by the anterior corneal surface of an eye, will be collected by the objective lens assembly of the tonometer, to appear as two areas of light in the field of view.

Preferably the spacing and position of the two light sources relative to the objective lens assembly are selected so that as the unit is moved towards an eye under test and approaches the critical distance from the eye at which the pupil image disappears, the light reflected by the corneal surface will appear as two closely spaced spots of light which, with continued movement of the unit towards the eye, will begin to move away from each other, to be replaced by two areas of light corresponding to the mask windows as the unit approaches the critical firing distance from the eye.

Seeing the two spots of light in the field of view, ahead of the two areas of light relating to the masks, assists the user in knowing that the unit under his/her control is approaching the eye under test but still needs to be moved towards the patient.

The light from each source may be coloured and distinct from the colour of that from the main source of illumination.

Where the source of illumination is red, a preferred colour for light from each of the two small supplementary sources is green.

In addition, the position of the two spots of light relative to the centre of the field of view will also tell the user whether the unit is centred on the eye. Thus if the spots are not symmetrically located about the centre of the field of view, and do not lie on a straight line passing through that central region of the field of view, the optical axis of the unit is probably not centred on the eye. Movement of the unit to the left or the right (and/or up or down if the spots are too low or too high) will attain the desired adjustment, enabling the user to then move the unit in a forward direction in the knowledge that it is correctly centred on the eye under test.

Preferably the two small light sources are positioned so that light therefrom is directed towards the anterior corneal surface of the eye, such that when the latter is at a distance from the tonometer which is just greater than the critical distance at which firing will occur, two distinct spots of light will be visible in the field of view and will move apart and disappear and be replaced by the light from the main source of illumination which illuminates the two mask windows as the unit is moved closer to the eye.

Preferably the light from these two light sources is of a different colour from the other light images which appear in the field of view during use.

In particular it is very desirable that the wavelength of the light from each small light source is significantly different from that of the main source of illumination, and the photo-sensors are selected so as to have a peak response to the wavelength of the light from the main source and a minimal or zero response at the wavelength of light from each supplementary light source, so that light from the latter which may reach the photo-sensors does not significantly affect the output thereof.

Typically the two small sources comprise two LED's .

Preferably lens-capped LED's are used the focusing effect of the integral lenses serving to concentrate the light therefrom towards the eye under test. If the LED's do not include integral lens caps, separate miniature lenses may be provided to focus the emitted light as required.

Power for the LED's may be obtained from a power supply associated with the tonometer unit.

An ON/OFF switch may be provided to power the LED's only when required.

Such a switch may be operated by a push button on the unit, located so as to be capable of being pressed by the thumb or a finger of the hand used by the user to hold the tonometer.

Preferably power to the LED's is removed upon the firing of the unit, and the ON/OFF switch may be associated with or be integrated into the RESET switch associated with the unit, which has to be pressed to arm the unit ready to detect an eye and fire an air pulse towards it.

Alternatively the two light sources may comprise two optical fibres leading away from a lamp in the tonometer.

If coloured light is required the optical fibres may be formed from coloured glass or the light path may include a coloured filter.

Preferably the lamp is the filament lamp used to illuminate the mask in the objective lens assembly, with the light for the fibres being obtained from upstream of the red filter.

If angled semi-reflecting surfaces are employed in the tonometer optics, then the two windows of the mask need to be oriented so that the optical path to the photodetectors is the same for each window so that both will be imaged in the same way at the same time.

In order for the light from the supplementary sources to shine through the two windows, following reflection from the patient's cornea, and be seen by the user of the tonometer it is preferable for the supplementary sources to be oriented in a plane going through the centre of the two windows.

Preferably therefore, where the plane semi-reflecting mirrors are angled about a horizontal axis (that is when the tonometer is held upright), the two points are to the left and right of the objective lens assembly and the LED's or fibre optic ends may be incorporated into the tonometer housing or in lateral enlargements on either side of the tonometer housing.

The invention will now be described by way of example with reference to the accompanying drawings, in which:

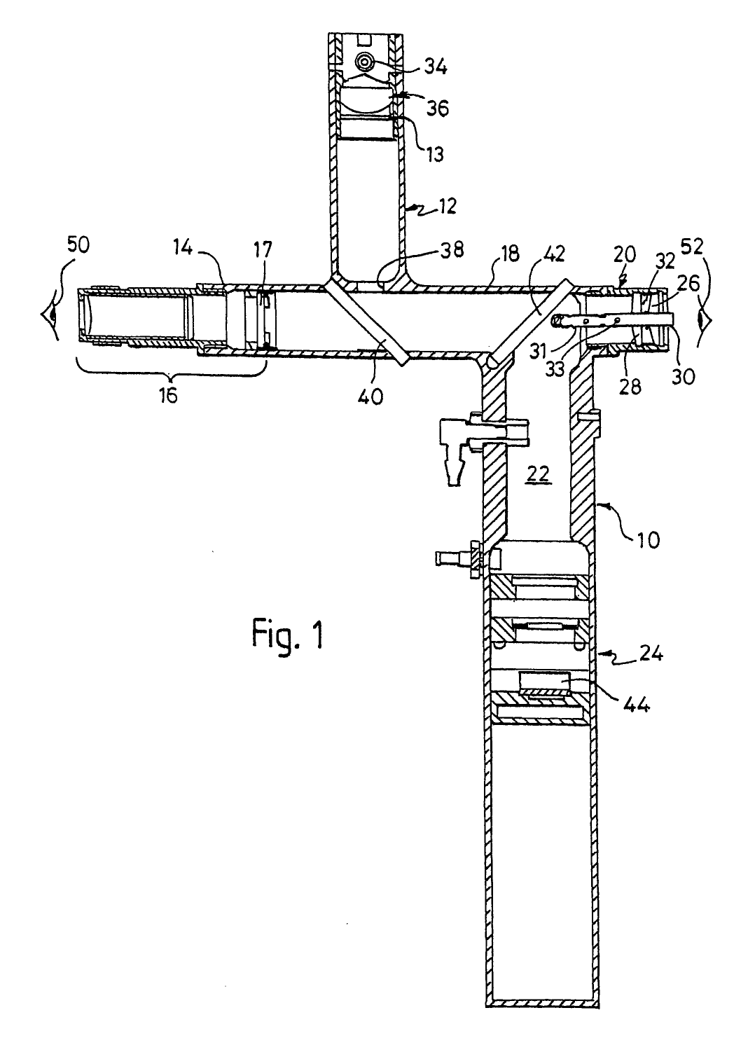

FIG. 1 is a cross-section through the optics and pneumatic chamber of an air impulse tonometer of the type described and can be compared with the drawings in UK 2175412 and EP 0289545,

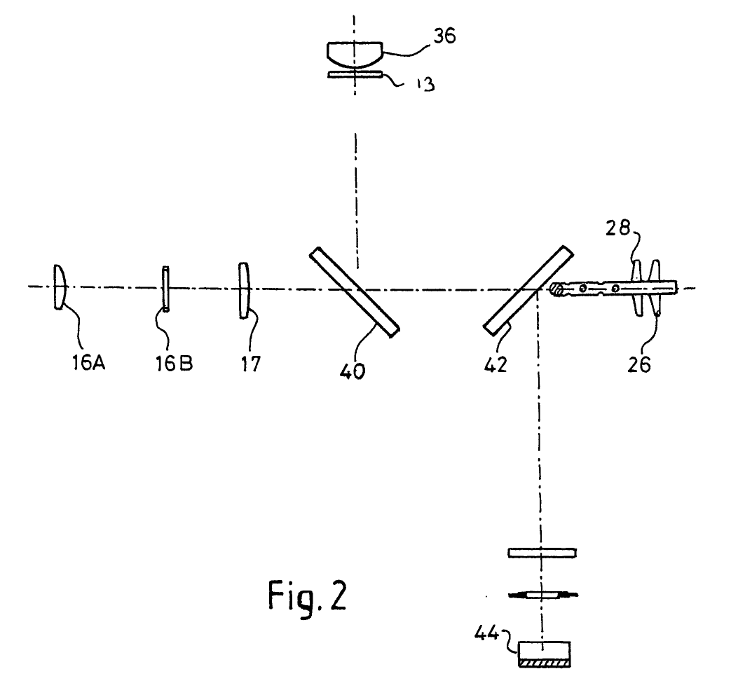

FIG. 2 is a schematic of the optical paths of the device shown in FIG. 1,

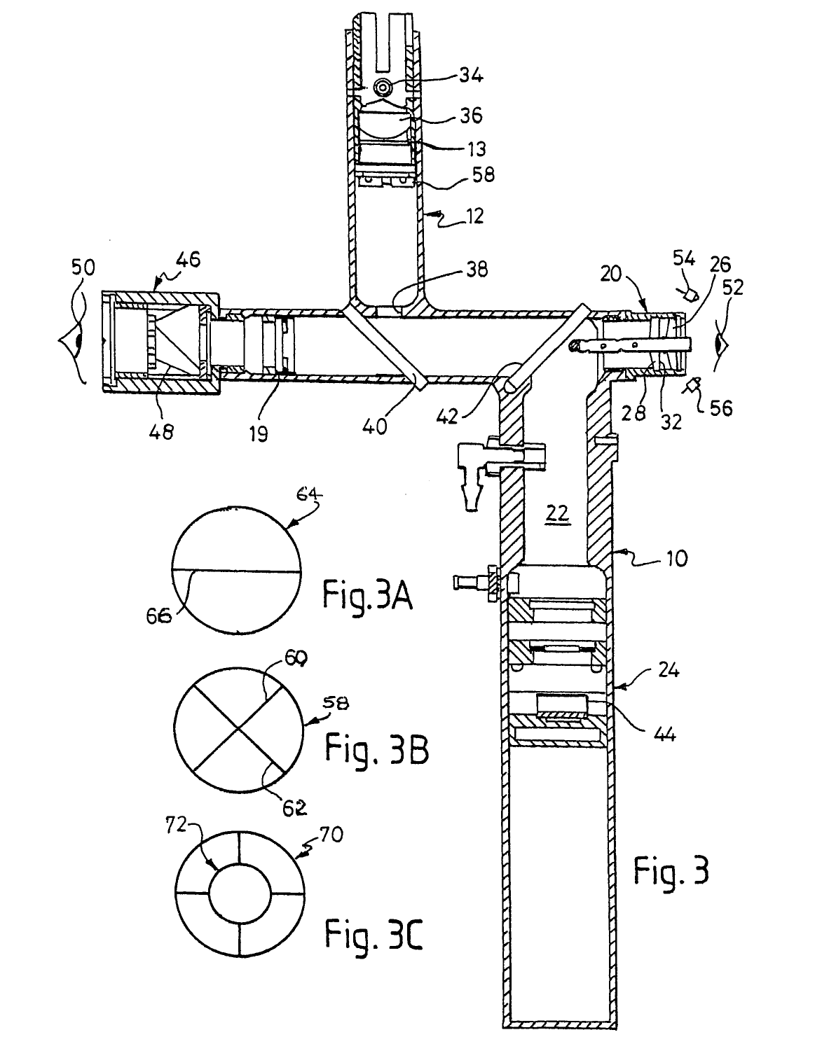

FIG. 3 is a cross-section through an air impulse tonometer similar to that of FIG. 1, but modified to incorporate the present invention and incorporating modified eyepeice which includes a roof-prism to invert the image,

FIGS. 3A-3C show different hair-line objects for inclusion in the lamp housing,

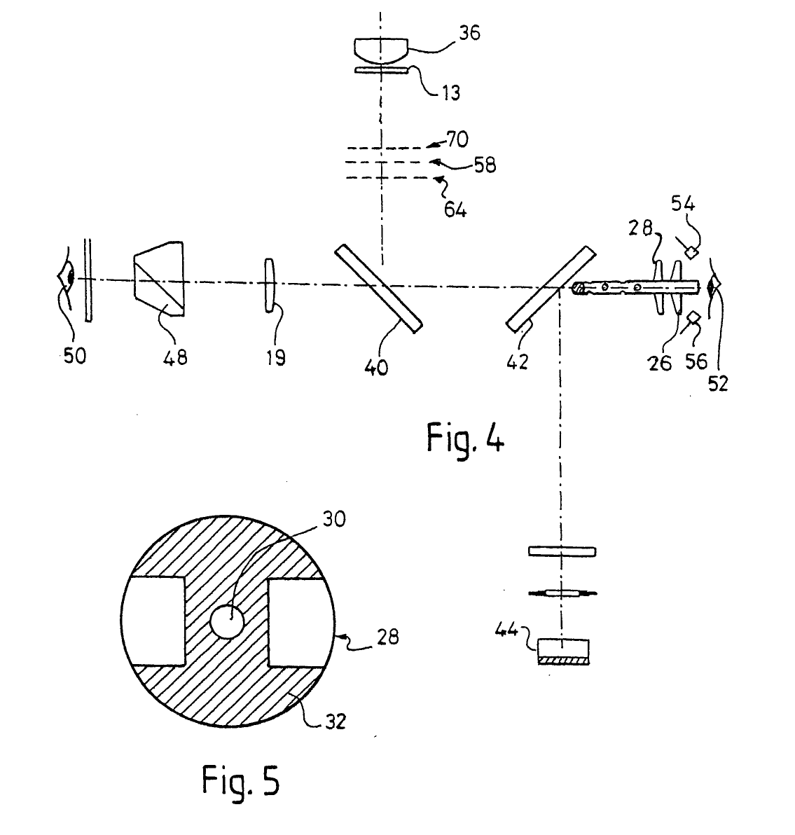

FIG. 4 is a schematic of the optical paths of the device shown in FIG. 3, and

FIG. 5 shows the form of the mask on one of the lenses in the final lens assembly,

As shown in FIGS. 1 and 2 a machined chassis 10 comprises a lamp housing 12, a viewing end 14 containing an eyepiece 16 containing a lens 16A, and field stop 16B and field lens 17 (see FIG. 2), a beam splitting section 18, nozzle 20, a plenum chamber 22 and a sensor chamber 24. The tube 20 contains an objective lens assembly 26, 28 and central puff tube 30 supported by the lenses 26, 28 through which it extends. A filter 13 restricts the light transmitted downstream therefrom to wavelengths in the red/infra-red range of the spectrum.

A mask 32 is screen printed onto the face of lens 28, the form of the mask being shown in FIG. 5, as it will appear if viewed axially of the puff tube. The mask has two windows but is otherwise opaque.

The lamp housing 12 includes a filament bulb 34 from which light is projected as parallel light by a condensing lens assembly 36 to illuminate an aperture 38 at the junction of the housing 12 and the beam splitting section 18. Light passing through 38 is reflected by semi-reflecting mirror 40 towards another semi-reflecting mirror 42 through which it can pass and be focused by the objective lenses 26, 28 onto an eye under test 52. A fraction of the light reflected by the eye and collected by the objective lenses 26, 28 will be reflected by mirror 42 into and through the plenum chamber 22 towards a photoelectric detector assembly 44 in the sensor chamber 24. The remainder will travel through the semi-reflecting mirror 42 and on through the semi-reflecting mirror 40, to the eyepiece 16.

The field lens 17, typically having a focal length of the order of 62 mm, co-operates with the lens 16A in the eyepiece to form an in-focus view of the image of the mask 32 which is formed from the convex curvature of the patient's cornea and the objective lenses 26, 28 to an observer viewing through the eyepiece 16. The lens 16A typically has a focal length of 25 mm. The presence of the mask and puff tube means that the image of the mask, reflected by the patient's eye 52 will, when correctly focused, appear as two segments, each similar to a capital letter D, one being a mirror image of the other. The in-focus condition will only occur when the eye is at a particular distance from the end of the puff-tube 30 determined by the focal length of the objective lens assembly 26, 28, and the radius of curvature of the patient's cornea. Typically each of the lenses 26 and 28 is a plano-convex lens having a focal length of the order of 40 mm.

The plenum chamber 22 is pressurised with air when a pulse of air is required. Ignoring the passage leading to the pressure transducer (not shown) the chamber 22 is closed, and air can only escape via the tube 30. The air escapes as a single pulse, the leading edge shape and duration of which is dictated by the geometry of the tube 30 and openings 31, 33, the volume of the plenum chamber 22, the shape and volume of the passage leading to the pressure transducer (not shown), and the volume of the pulse of air introduced into the plenum chamber. As described in GB 2175412 and EP 0289545 the exact point in time when a pulse of air is released into the plenum chamber to create a pulse of air through the puff tube, is controlled by a control system (not shown) triggered when an appropriate pattern of light falls on the photodetectors in the sensor chamber 24.

The essential elements of the optical system of FIG. 1 are shown in FIG. 2, where the lenses and field stop making up the eyepiece 16 are denoted as 16A and 16B and 17.

FIG. 3 shows how the arrangement of FIG. 1 can be modified in accordance with the present invention.

In accordance with the invention an object in a supporting frame or transparent substrate 58 comprising cross hairs 60, 62 (see FIG. 3B), is located downstream of the filter 13 in the lamp housing 12.

The position of the object 60, 62 in the support 58 is selected so that the image of the cross hairs 60, 62 comes into focus for the operator at the same distance from the objective lenses to the patient's cornea as gives a correctly aligned and in focus image of the mask 32 onto the plurality of photodetectors 44.

A second object 64 (see FIG. 3A) may be located downstream of 58 containing a single cross hair 66, which will come into focus just before the cross hairs 60, 62.

A third object 70 (see FIG. 3C) containing a different array of cross hairs such as 72, may be located upstream of 58. The visible parts of this object will appear and come into focus if the unit is moved closer to the eye. Continued movement towards the eye can cause the lamp filament to appear and come into focus. Preferably the diameter of the circular wire loop in the array 72 is large enough for parts of it to appear in the two illuminated windows of the mask.

The user can therefore be instructed to look for the cross hairs 66, 68 and watch for their replacement by hairs 60, 62 which, when in focus and centred in the field of view, will indicate that the unit should be at the critical distance from the eye 52 for firing to occur. If perchance the hairs 60, 62 are missed by the user and hair array 72 appears, the user will know to move the unit back, away from the eye, to look for hairs 60, 62.

To make the initial positioning of the tonometer relative to a patient's eye somewhat easier, the eyepiece 16 may be replaced with eyepiece 46 shown in FIG. 3 containing a single lens 19 having a focal length of the order of 80 mm. Lens 19 forms a simple telescope with the objective lenses 28, 26 which enables the operator to see the patient's eye from a distance. The eyepiece 46 as shown in FIG. 3 also contains a Pechan-Schmidt prism 48 (sometimes called a roof-prism). This presents a correctly orientated and handed image of the patient's face and eye to the user.

When using a modified eyepiece such as 46, a user no longer has to squint along the side of the unit to see if the unit is correctly positioned relative to the eye. Instead the user can now look through the eyepiece and see the face and eyes of a patient at a distance of say 0.5 m. The user can then move the unit so as to centre it on (say) the right eye of the patient and then move forward keeping that eye in the centre of the field of view and centred on the pupil of that eye. As the unit is moved nearer to the eye, the pupil image becomes larger and shortly before or after it fills the field of view so that the latter becomes dark and to this end a further aid is provided in the form of two small supplementary light sources, the reflections of which form two spots of light in the filed of view initially close together and near the centre of the field of view.

Continued forward movement will cause the two green spots to move outwards in opposite directions and disappear, thereafter to be followed by red light which appears as two spaced apart distinct red areas centrally of the field of view and which with continued forward movement enlarge and fill the windows of the mask in the field of view.

The two supplementary light sources are shown in FIG. 3as two green LED's 54,56 are located one on each side of the puff tube 30 directed towards the patient's eye 52 and equally spaced from the puff tube and objective lens axis. Although as depicted in FIG. 3 the LED's are shown apparently above and below the puff tube 30, they are more preferably mounted to the left and right of the puff tube 30. The reflections of the two LED's in the eye appear as two green spots in the field of view of the eyepiece lens 19.

Thus, with continued forward movement of the unit, the operator will see two small green spots which then move apart. Then just as the spots begin to disappear to the left and right of the field of view the red light from 34, 36 which has been reflected from the patient's cornea, begins to illuminate the windows of mask 32 and appear in the field of view.

The green light spots therefore represent an advance warning that the red light will shortly appear and if they do not appear symmetrically about the centre of the field of view, the user knows that the unit is not positioned correctly relative to the eye, and can move it accordingly.

As the critical distance from the eye is reached, the black image of the wires 60, 62 of object 58 appear in the otherwise red field of view and come into focus at the precise position at which firing will be triggered. If objects 64 and 70 are also fitted, one of these will appear and come into focus and then go out of focus and disappear just before the wires 60, 62 of 58 appear and come into focus. The wire(s) of the other object will only appear if the unit is moved through the critical position, so as to be too close to the patient's eye. Continued movement of the unit towards the eye will result in the filament of the bulb 34 coming into focus.

If the unit is not centred on the eye, the crossing point of the two wires 60, 62 will not coincide with the centre of the field of view and the wires will appear asymmetrical relative to the field of view. Movement of the unit up or down or sideways to correct this, will find the correct position at which the unit will fire.

It is to be understood that the term lens employed herein can mean a single or multiple element lens.

In addition the colour of the light from the two supplementary light sources may be the same, or different. Thus, if the main source is red, one supplementary source may be green and the other for example yellow or blue.

1-31. (cancelled).

32. A hand-held air impulse tonometer which includes an objective lens and puff tube assembly and an eyepiece through which a user looks, and in which there is an object at a point in an optical path of light from a source of illumination in the tonometer such that an in-focus image of the object will be formed in the user's field of view, as seen through the eyepiece, when the tonometer is at a critical distance from an eye under test at which an automatic air pulse generating means will be triggered by light reflected from the eye.

33. A tonometer as claimed in claim 32, wherein the object is located near to the source of illumination.

34. A tonometer as claimed in claim 32, further comprising a second object located within the same region of the tonometer as the first object and visually distinguishable from the first object, but in a plane which is spaced from the plane containing the first object, whereby its visually distinguishable image will come into focus in the field of view just before the image of the first object comes into focus, as the unit is moved towards the patient's eye.

35. A tonometer as claimed in claim 34, further comprising a third object also located within the same region and visually distinguishable from both the first and the second objects and located in a third plane spaced from the plane containing the first object, whereby its visually distinguishable image will come into focus if the unit is moved closer to the patient's eye than said critical distance.

36. A tonometer as claimed in claim 34, wherein each object comprises a single line, and the images of the lines appear at different orientations when viewed through the eyepiece.

37. A tonometer as claimed in claim 32, wherein the object is an opaque hairline pattern in a transparent support.

38. A tonometer as claimed in claim 32, wherein the object is a pattern etched from a metal film on a sheet of glass or plastics material or an etched metal foil, the etching defining a pattern.

39. A tonometer as claimed in claim 37, wherein the pattern comprises at least one line which extends in a plane generally perpendicular to the axis of the light path.

40. A tonometer as claimed in claim 32, wherein the object is planar, and wherein a pattern exists in the object and comprises a single line, two lines which cross at an angle, a circle with at least two radii, or a spiral.

41. A tonometer as claimed in claim 32 in which the eyepiece lens assembly and the objective lenses form a simple telescope to give an in-focus image of the patient's eye in the user's field of view when the latter is at a distance from the tonometer, to allow the user to look through the eyepiece and identify the patient's eye to be tested, while the tonometer is distant from the patient's face.

42. A tonometer as claimed in claim 41 wherein the eyepiece includes a Pechan-Schmidt prism to invert the image of the patient's eye and thereby present to the user an image of the eye which is correctly oriented and handed in a vertical and horizontal sense.

43. A tonometer as claimed in claim 41, wherein the focal length of the eyepiece is in the range 62-100 mm.

44. A tonometer as claimed in claim 32, further comprising two supplementary light sources at diametrically opposite points from the optical axis of the objective lens assembly of the tonometer, such that light from the two sources, after reflection by the anterior corneal surface of an eye under test, when close to the eye, will be collected by the objective lens assembly of the tonometer, to appear as two areas of light in the field of view, the spacing and position of the two supplementary light sources relative to the objective lens assembly being selected so that as the unit is moved towards an eye under test and approaches the distance from the eye at which the image of the pupil fills the user's field of view and the image of the eye disappears, the light reflected by the corneal surface will appear as two closely spaced spots of light which, with continued movement of the unit towards the eye, will begin to move away from each other, to be replaced by two other areas of light as the unit approaches the said critical distance.

45. A tonometer as claimed in claim 44, wherein the light from the two supplementary sources is coloured differently from that forming the said two other areas.

46. A tonometer as claimed in claim 44, wherein the points are equidistant from the said optical axis.

47. A tonometer as claimed in claim 44, wherein the wavelength of the light from each supplementary light source is significantly different from that forming the said other areas, and the photo-sensors of the tonometer detector are selected so as to have a peak response to the wavelength of the other light and a minimal or zero response at the wavelength of light from each small light source so that light from the latter which may reach the photoelectric sensors does not significantly affect the output of the sensors.

48. A tonometer as claimed in claim 44, wherein each supplementary source comprises an LED.

49. A tonometer as claimed in claim 44, wherein the two supplementary light sources comprise two optical fibres arranged to convey light away from a lamp in the tonometer.

50. A method of measuring the intra-ocular pressure of a patient's eye using a tonometer as claimed in claim 44, wherein lenses in the eyepiece and the objective form a simple telescope to give an in-focus image of the patient's eye in the user's field of view, and wherein the user looks through the eyepiece to select the patient's eye to be tested whilst at a distance from the patient's face, and thereafter moves the unit towards the eye, keeping the image of the eye in the centre of the field of view, and centred on the eye after the user's field of view is completely filled by the image of the pupil, and continuing to move the tonometer closer towards the patient's eye so that two spots of light originating from the two supplementary light sources, and reflected off the patient's eye appear midway of the field of view and then diverge as the tonometer is moved still closer to the eye, until light from the source of illumination in the tonometer which is of a colour different from that of light from the two supplementary sources and is projected by the tonometer towards the eye under test, is reflected by the eye to appear as two areas of that different colour which replace the diverging spots of light and which with continued movement of the tonometer towards the eye, enlarge to at least partly fill the field of view indicating the tonometer is close to the position at which the pneumatic air puff generating system of the tonometer will be automatically triggered to discharge a puff of air towards the eye, and thereafter moving the tonometer closer to the eye so as to bring into sharp focus an image of the said object, at which point the air puff is discharged towards the eye, and a numerical value proportional to the intra-ocular pressure of the eye is computed from signals indicative of a variation in the light reflected by the eye and received by a detector in the tonometer as the eye is momentarily distorted by the force of the puff of air.

51. A method of measuring the intra-ocular pressure of a patient's eye as claimed in claim 50 wherein the computed numerical value is displayed by the tonometer.
